(19) United States
(12) Patent Application Publication
(10) Pub. No.: US 2024/0058588 A1
(43) Pub. Date: Feb. 22, 2024

(54) 3D PRINTED MEDICAL BALLOONS AND METHODS OF INTEGRATING HIGH STRENGTH FIBERS IN MEDICAL BALLOONS

(71) Applicant: Abtihal RAJI-KUBBA, Phoenix, AZ (US)

(72) Inventor: Abtihal RAJI-KUBBA, Phoenix, AZ (US)

(21) Appl. No.: 18/268,187

(22) PCT Filed: Dec. 17, 2021
(86) PCT No.: PCT/US2021/072982

§ 371 Date: Jun. 16, 2023

Related U.S. Application Data
(60) Provisional application No. 63/127,889, filed on Dec. 18, 2020.
(60) Provisional application No. 63/138,311, filed on Jan. 15, 2021.
(60) Provisional application No. 63/165,019, filed on Mar. 23, 2021.

(51) Int. Cl. A61M 25/10; B33Y 80/00; B33Y 10/00; B29C 64/118

(52) U.S. Cl. CPC A61M 25/1029; B33Y 80/00; B33Y 10/00; B29C 64/118; A61M 2025/1031; A61M 2025/1084; A61M 2025/1079; B29L 2031/7543

(57) ABSTRACT

Some examples of the disclosure are directed to medical balloon comprising a balloon base having an interior surface and an exterior surface, wherein the balloon base includes a base material (105), and one or more fibers (107) embedded within the base material. Some examples of the disclosure are directed to a medical balloon comprising a balloon base having an interior surface and an exterior surface, wherein the balloon base includes a base material (205), and one or more 3D printed fibers (207a) on a respective surface of the balloon base. Some examples of the disclosure are directed to manufacturing methods for forming one or more medical balloons.

### CROSS-REFERENCE TO RELATED APPLICATIONS

This application claims priority to U.S. Provisional Patent Application No. 63/127,889, filed Dec. 18, 2020, U.S. Provisional Patent Application No. 63/138,311, filed Jan. 15, 2021, and U.S. Provisional Patent Application No. 63/165,019, filed Mar. 23, 2021, the entire disclosures of which are incorporated herein by reference for all purposes.

### FIELD OF THE DISCLOSURE

This relates to medical balloons with integrated fibers, and methods for manufacturing such medical balloons.

### BACKGROUND OF THE DISCLOSURE

High-pressure balloons have traditionally been used in angioplasty—a procedure that opens a blood vessel clogged by a buildup of fatty plaque. Innovations in balloon design and technology have provided increased flexibility to product designers, making the development of new and improved devices possible. As a result, high-pressure balloons are being employed in a growing number of diagnostic and therapeutic procedures. Applications include the following: angioplasty catheters, other dilatation catheters, stent-delivery catheters, heat-transfer catheters, photodynamic therapy devices, laser balloon catheters, and cryogenic catheters, such as described in an article by Mark A. Saab in Medical Device & Diagnostic Industry Magazine —originally published September 2000.

### SUMMARY

Some examples of the disclosure are directed to medical balloon comprising a balloon base having an interior surface and an exterior surface, wherein the balloon base includes a base material, and one or more fibers embedded within the base material. Some examples of the disclosure are directed to a medical balloon comprising a balloon base having an interior surface and an exterior surface, wherein the balloon base includes a base material, and one or more three-dimensionally (“3D”) printed fibers on a respective surface of the balloon base. Some examples of the disclosure are directed to manufacturing methods for forming one or more medical balloons.

### BRIEF DESCRIPTION OF THE DRAWINGS

For a better understanding of the various described embodiments, reference should be made to the Description of Embodiments below, in conjunction with the following drawings in which like reference numerals refer to corresponding parts throughout the figures.

FIG. 1 illustrates a cross-section of an example medical balloon of the disclosure that includes fibers within a balloon base according to examples of the disclosure.

FIG. 2 illustrates a cross-section of an example medical balloon of the disclosure that includes fibers on an exterior surface of a balloon base according to examples of the disclosure.

FIG. 3 illustrates a cross-section of an example medical balloon of the disclosure that includes multiple layers of fibers on an exterior surface of a balloon base according to examples of the disclosure.

FIG. 4 illustrates a perspective view of a medical balloon according to examples of the disclosure.

### DETAILED DESCRIPTION

In the following description, reference is made to the accompanying drawings which form a part hereof, and in which it is shown by way of illustration specific examples that can be practiced. It is to be understood that other examples can be used and structural changes can be made without departing from the scope of the examples of the disclosure.

FIG. 4 illustrates an example of an exterior view of a medical balloon, such as a medical balloon of the disclosure. The balloon (e.g., 401), tightly wrapped around a catheter shaft (e.g., 403) to minimize its profile, can be inserted through the skin and into the narrowed section of the vessel. Inflating the balloon—typically with saline or a radiopaque solution forced through a syringe—exerts high pressure to the vessel, which compresses the plaque against the wall of the vessel and allows the blood to flow normally. For retraction, a vacuum is pulled through the balloon to collapse it. The procedure was developed as a less-invasive and less-costly alternative to coronary bypass, a complex surgical procedure that skirts the blockage by grafting a section of vein, usually taken from the leg, to locations above and below the afflicted area. A medical balloon of the disclosure can also be used to aid in the delivery of other medical devices; for example, to expand a stent during delivery and deployment into a narrowed vessel.

Balloons can be rated by the average pressure at rupture. Some examples of non-compliant fiber reinforced balloons, also known as ultra-high-pressure balloons, and methods for manufacturing them are described in U.S. Pat. No. 6,746,425. For example, U.S. Pat. No. 6,746,425 describes a (e.g., standard) polyethylene terephthalate (“PET”) balloon with multiple (usually 2) layers of high strength inelastic fibers each of which is set manually in a patterned layer. The first layer is set in a longitudinal direction relative to the balloon y axis and is bonded by adhesives on the base balloon. Adhesive is applied on top of the first layer of fibers before a second layer of fibers is set in a radial direction on the x axis of the balloon. Variations of fiber laying methods and patterns were further described in U.S. Pat. No. 8,002,744 (and others) aimed at simplifying the manual construction of and reducing the time and labor required to manufacture these balloons and their defect rate. Due to the multiple layer construction of these balloons, they can be relatively thick and stiff which can make it difficult to wrap them tightly when deflated. This can be a disadvantage as the finished product can have an undesirably high profile (e.g., profile is the maximum diameter of the balloon when mounted on a catheter in its deflated and wrapped condition or the smallest hole through which the deflated balloon catheter can pass).

Some examples of the disclosure are directed to three-dimensional (“3D”) print manufacturing of and/or 3D printed fiber reinforced and/or integrated non-compliant medical balloons. The final balloon material can be a matrix of a polymeric material (e.g., the base material(s)) and fibers. Some examples of the disclosure are directed to an ultra-high-pressure non-compliant medical balloon design suitable for angioplasty and other medical procedures and which can integrally include high tensile strength fiber material (such as Kevlar, Dyneema, ultra-high molecular weight polyethylene and the like) encased and/or embedded in the base material, manufactured using 3D printing methods. The final balloon material can be a matrix of a polymeric material and fibers. The addition of high strength fibers such as Kevlar, Dyneema, UHMWPE, and the like can add structural strength to the base balloon. Some examples of the disclosure utilize a 3D printing process that eliminates the need for currently known costly, labor intensive and time-consuming manual fiber laying assembly designs and methods.

As mentioned previously, some examples of the disclosure are directed to 3D print manufacturing of fiber reinforced and/or integrated non-compliant medical balloons=. In a material extrusion 3D printing process, the extruder can be heated, which can liquify the thermoplastic build material, which can start to cool down as soon as it exits the printer nozzle. The cooling of the freshly deposited material can happen through convection with the surrounding air and through conduction with the previously deposited material.

Additive manufacturing systems such as fused deposition modeling 3D printing are typically equipped with three linear motion axes to position the processing tool head relative to the building platform in a translational motion. The process paths for each slice are planned based on slicing a CAD model of the part. The path is computed by the slicer software, which allows users to change certain print parameters, such as print speed, nozzle temperature, or infill pattern. Therefore, 3D printing provides a high degree of design optimization and integration of features and enables flexibility in manufacturing batch sizes.

In at least some aspects of this disclosure, relevant melting temperatures are considered in the selection of fiber material for pairing with the base balloon material. This thermal compatibility serves to preserve the integrity of the fiber material structural strength during processes such as extrusion and printing.

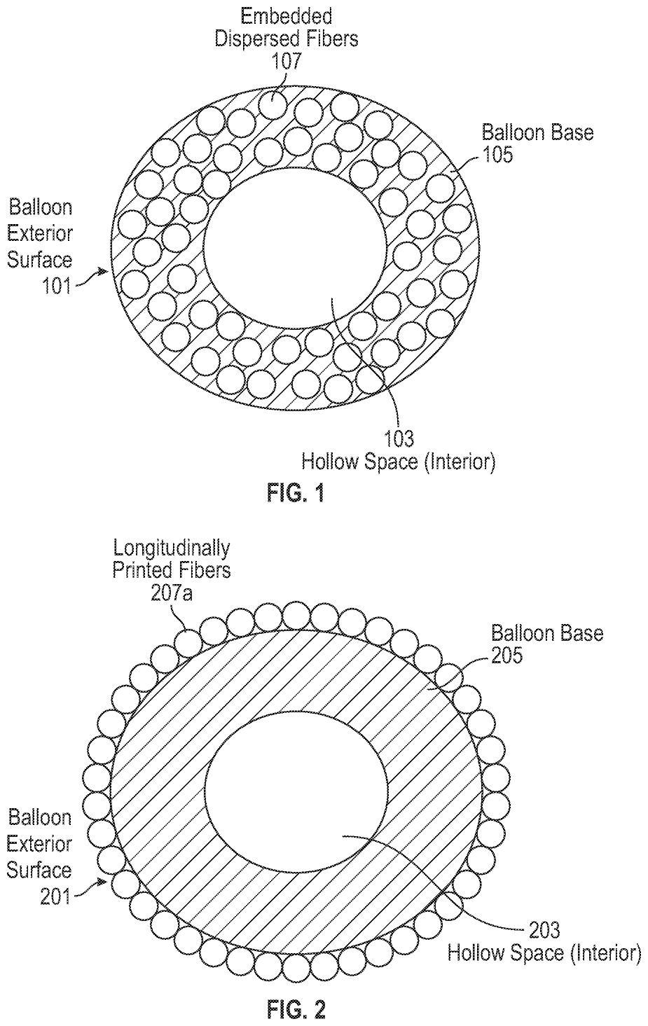

FIG. 1 illustrates an example cross-section of a medical balloon in accordance with some examples of the disclosure. For example, FIG. 1 illustrates a cross-section of a fiber-balloon matrix according to examples of the disclosure. The balloon of FIG. 1 can comprise a base material 105 (e.g., as described herein) having a substantially circular cross-section as shown. The balloon can have an exterior surface 101 (e.g., with a substantially circular cross-section), a hollow interior volume 103 (e.g., with a substantially circular cross-section), and one or more fibers 107 embedded within the base material 105. In particular, high strength fibers such as Kevlar or Spectra as examples, can be blended (compounded), at a specified temperature, rotor speed and % wt. fiber, in a base thermoplastic material such as PET to create a feedstock composite filament matrix which can then be extruded and used (e.g., by a 3D printer) for 3D printing balloons using a commercial desktop 3D printer. Mixing or filling a thermoplastic matrix (e.g., such as the one previously mentioned) with short fibers in base materials can be utilized. In some examples, the compounding temperature is based on (e.g., is that of) the selected thermoplastic and/or fiber combination. For example, for PET, the compounding temperature can be 250-260 degrees Celsius (e.g., corresponding to the melting temperature of PET). In some examples, the rotor speed for compounding can be based on the selected thermoplastic and/or fiber combination—in some examples, this speed can be around 40-60 rpm. In some examples, the % fiber content of the compound can be selected to achieve a high (e.g., highest) packing density-strength for the design of the balloon and the application and/or to reduce or minimize voids in the compound. In some examples, the % wt fiber can be 1%, 5%, 10%, 20%, 30%, 40% or 50%, or 1-50%. In some examples, the fiber diameter used can be 5 microns, 7 microns, 10 microns, 15 microns or 5-15 microns. In some examples, the fibers can be etched (e.g., physically, chemically and/or electrically) prior to compounding to increase (e.g., maximize) bonding between the base material and the fibers. Studies on carbon fibers in polymer composite have documented that fiber reinforcement enhances the properties of resin/polymeric matrix materials. Specifically, 3D printing not only yields very high fiber orientation in the printing direction but also molecular orientation and, hence, increased tensile strength for the balloon.

In some examples of the disclosure, the 3D printed medical balloon extrusion membrane tensile strength and hoop stress can be varied (e.g., from one balloon design to another) by varying the fiber content and/or orientation in the blended matrix resin. In some examples, the direction of printing of the fibers can be within a threshold orientation (e.g., 0, 1, 3, 5, 10, 20, 30 or 45 degrees) of the predicted load-bearing direction and/or fail-safe direction of the completed medical balloon. Additional or alternative specifications can be articulated in terms of optimum fiber-in-resin packing density (ratio of volume of fibers/volume of fibrous medium or base), and homogeneous pore-size distribution. Additionally, tensile strength and/or hoop stress can be varied by the extrusion path (nozzle angle) during the printing process and/or balloon wall thickness varied by the extrusion nozzle diameter. In some examples, the nozzle angle can be 0 degrees, 30 degrees, 45 degrees, 60 degrees or 90 degrees, or anywhere from 0 to 90 degrees during printing.

In some examples, the printed base material 105 and fiber 107 layer illustrated in FIG. 1 can be partially or fully repeated (e.g., additional printing layered on the partial or full exterior surface 101 and/or interior surface 103), such as for added strength and/or other functionality. In such examples, prior to such repeated printing, the exterior surface 101 and/or the interior surface 103 can be etched (e.g., physically, chemically and/or electrically) to enhance bonding of such additionally printed layers. In some examples, additional base material 105 and/or thermoplastic material (e.g., not including fibers 107) can be printed/layered on exterior surface 101 and/or interior surface 103.

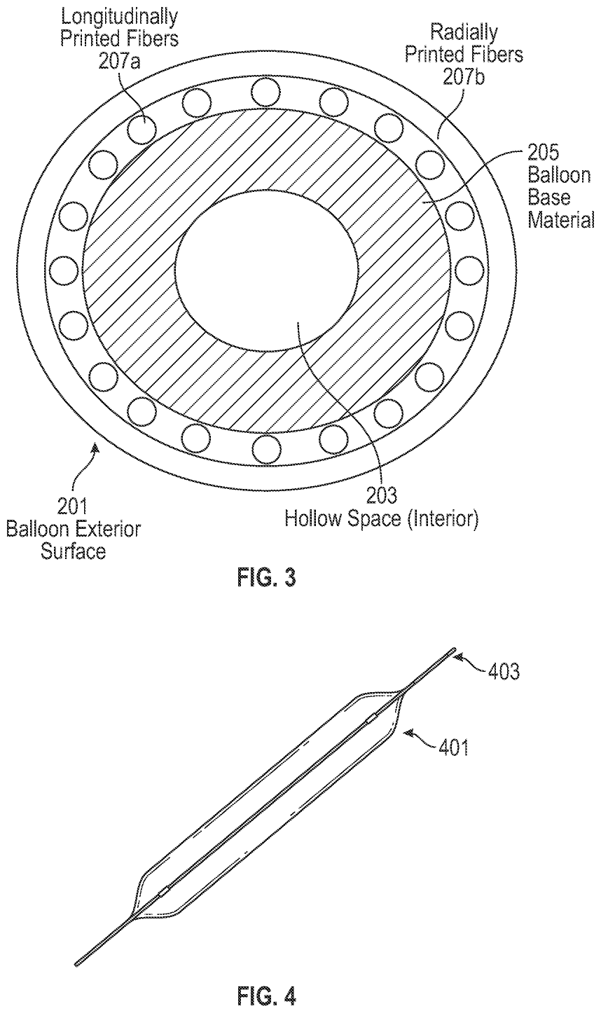

In some examples of the disclosure, the medical balloons of the disclosure can be formed by 3D printing continuous (not short) fiber reinforced thermoplastic material or filament. In such examples, there is no need to print a base layer of, for example, thermoplastic onto which a (e.g., continuous) fiber filament layer(s) is printed (e.g., such as in FIGS. 2-3). Rather, in such examples, the continuous fiber reinforced thermoplastic material or filament (e.g., concentric circles of continuous fiber core embedded in and surrounded by thermoplastic) can be fed from a 3D printing nozzle, and printed in a pattern to form the structure of the medical balloon (e.g., without another material being fed from that nozzle or another nozzle). In some examples, the continuous fiber filament can be fed from a first 3D printing nozzle alternating with a second 3D printing nozzle that feeds the thermoplastic material, the two nozzles together printing their respective materials in a pattern to form the structure of the medical balloon. In another example, the thermoplastic material is fed through the nozzle while continuous fiber yarn is mechanically fed and laid onto it.

In some examples of the disclosure, continuous fiber layers can be embedded onto a previously formed base PET nylon balloon, or other thermoplastic material, and can be achieved by adapting continuous fiber laying automation techniques of a fiber laying process, such as a composite 3D printing process described in U.S. Pat. No. 9,956,725, which utilizes continuous carbon fiber strands or fiberglass strands embedded into a 3D printed nylon substrate. FIGS. 2 and 3 illustrate examples of such embodiments, which both illustrate cross-sections of example medical balloons having hollow interior volumes 203 (e.g., corresponding to interior volume 103), base material 205 (e.g., corresponding to base material 105) and exterior surface 201 (e.g., corresponding to exterior surface 101). In some examples, base material 205 has one or more of the characteristics of base material 105 (e.g., including one or more of the fibers referenced in this disclosure)—in some examples, base material 205 does not include fibers.

Embedding of select core reinforced fiber filament materials in a thermoplastic base material can be feasible due to the melting temperature of fibers such as Kevlar, among others, being higher than that of PET, for example. In some examples, different fiber layering prints and patterns on the first/previously deposited base PET nylon balloon 205, or other material, are proposed. Continuous core reinforced fiber filament material 207a can be pack printed (minimal-no voids) or deposited at a desired thinness and/or pattern to achieve a lowest or reduced balloon profile and/or deposited at pre-specified spacing or density, such as a minimum of 1 fiber/mm, to achieve a desired strength on the previously deposited balloon base 205 surface in one direction such as a longitudinal direction (e.g., along the balloon length), such as shown in FIG. 2. In some examples, the printed core reinforced fiber filament material can be layered, with a first layer 207a being printed in a longitudinal direction, on which a second layer of fibers 207b can be weaved between or on the first layer (e.g., using the herein described printing) in a horizontal (e.g., radial or circumferential, around the diameter of the balloon) direction, such as shown in FIG. 3, among other possible layering sequences and patterns. In some examples, the fibers that are printed in the designs disclosed herein (e.g., the designs referenced in FIGS. 2 and 3) can be printed in straight, curved, or zig zag lines of equal or different thicknesses/widths and/or with or without breaks to create various target patterns. In some examples, prior to printing the second layer of fibers 207b on the first layer of fibers 207a, the surface of the first layer of fibers 207a can be etched (e.g., as described herein) or otherwise modified to prepare the surface of the first layer of fibers 207a for adhesion to the second layer of fibers 207b. In some embodiments, the directions in which the first layer 207a and the second layer 207b of the fibers are printed can be the reverse of what is described above. In some embodiments, fibers 207a and/or 207b can be printed on the interior volume 203 surface of base 205 rather than on exterior surface 201. In some examples, the base balloon material 205 in FIGS. 2-3 can be a pure thermoplastic or one containing fiber. In some examples, the printed balloon surface (e.g., 201) can be loaded with a therapeutic drug or a biologic.

The fibers (e.g., 207a, 207b) in FIGS. 2 and 3 can be etched to achieve surface chemical modification by, for example, gas plasma treatment, prior to or after the above-described fiber printing. Such etching using plasma(s) can improve the interfacial adhesion between fibers and matrix resins. Such etching can optimize fiber bonding with thermoplastic (e.g., 205) and/or facilitate drug loading on the surface (e.g., 201) of the balloon. In some examples, an outer layer of thermoplastic (e.g., on top of 207a and/or 207b) or an inner layer of thermoplastic (e.g., within interior volume 203) or another composite filler after fibers 207a and/or 207b have been printed can be added to the balloon.

In some examples, the spherically represented fibers in FIGS. 1, 2 and 3 could be flattened prior to or during any manufacturing step described herein to achieve better material packing, a lower profile balloon wall, an improved manufacturing process, and/or improved tube strength. For example, in some examples a mechanical fixture could apply compression tensioning to the fiber(s) during the feeding of the fiber yarn into the extrusion head for printing. In some examples, such flattening can be achieved by thermal forming by mechanically compressing and/or flattening the yarn to reshape it (e.g., ironing out the yarn between heated plates).

In some examples, the orientation of the deposited layers of fibers on such balloons (e.g., in FIG. 1, 2 or 3) can be selective such as to create a preferred topographical pattern on the balloon. A pattern can be programmed in the printing software to vary in any of the three axes (e.g., radial or other axes). Examples of a preferred pattern can be printing slanted longitudinal or radial layers of fibers (e.g., 207a and/or 207b)—such as at 10, 20, 30, 45, 60 or 75 degree angles relative to the axis defining the length of the balloon or the radius of the balloon—to facilitate and directionally bias the collapsing shape of a deflating balloon to minimize or bias its profile and avoid a flat “pan cake” shape at collapse, for example. Another example can be to create and print crease lines on base 205 to facilitate deflation of the balloon along a desired collapse path. A low-profile folded balloon allows fast and easy withdrawal of the balloon through a small sheath which is less traumatic at a vascular intervention site. In particular, a disorganized collapse of a balloon can make the balloon bulky and difficult to remove through the introducer. Therefore, in some examples, a path can be printed (e.g., in base 205 and/or on base 205 such as a path defined by 207a and/or 207b) to guide the balloon collapse following a preset route. One example of this kind of printing can be printing equally or unequally spaced wedges/tracks on or in base 205 at an extrusion/printer nozzle angle (e.g., left or right). Such printed features could serve as guide while folding the balloon in manufacturing (e.g., to a desired or preset orientation) and could bias the balloon to collapse back in that orientation and on that path during deflation.

In an example manufacturing method of the disclosure, the high-pressure medical balloon (e.g., as depicted in FIG. 1) can be manufactured using a co-extrusion method where continuous fiber yarn is fed from a spool or another fixture through a single die along with the base thermoplastic resin material such as PET so that the materials merge or weld together into a single structure before cooling. Fibers may be fed from one or more sources in longitudinal and/or radial and/or any direction along the three axes (e.g., radial or other axes). As used herein, fiber(s) may refer to a single strand, multiple strands, ribbon, woven, braided, or twisted forms of fiber. Fibers may be chemically etched (e.g., via plasma etching or etching methods such as wiping with or dipping the material in a chemical) prior to merging with the base thermoplastic resin material to enhance bonding. In some extrusion manufacturing methods of the disclosure, thermoplastic resin compounded with high strength fiber pulp such as Kevlar can be used to extrude the balloon tubing.

In another manufacturing method, the high-pressure medical balloon can be injection molded or formed using long fiber reinforced thermoplastic pellets or sheets. The latter can be made with a pultrusion process that impregnates continuous high strength fiber bundles, such as Kevlar, with resin such as PET, and then cuts them into long pellets. The continuous strands of fiber can be pulled through a die, where they can be coated and impregnated with thermoplastic resin.

For any and all of the designs and manufacturing methods disclosed herein, enhancing the balloon can be achieved by extra processing such as, for example, infusing a thermoplastic after printing by spraying, dipping, or coating the balloon tubing.

For any and all of the disclosed designs and manufacturing methods herein, in some examples, the minimum fiber thickness is optionally 5 microns, the minimum number of printed fibers is optionally 1 per mm, and/or the minimum combined fiber/thermoplastic wall/based thickness is optionally 30 microns.

For any of and all the disclosed designs and manufacturing methods herein, barium or barium sulfate (BaSO4) powder, which is efficient as an X-ray absorber, can be blended or added as a filler in the fiber reinforced thermoplastic resin (e.g., base material) or fiber filament to achieve increased x-ray radiopacity. For example, the powder can be blended with the fiber-filled thermoplastic filament or thermoplastic powder. In some examples, the powder comprises 4 to 20% powder by weight of the compound. Because the balloons of this disclosure incorporate fiber, the effect of the inclusion of the above-described powder in potentially lowering the tensile strength of the balloon can be offset. Visualization of the balloon under X-ray based on the inclusion of the above-described powder can eliminate the need for using contrast media solution while inflating the balloon, a step that slows down inflation and deflation times of the balloon due to the high viscosity of contrast media, adds cost, and procedure time.

Although examples have been fully described with reference to the accompanying drawings, it is to be noted that various changes and modifications will become apparent to those skilled in the art. Such changes and modifications are to be understood as being included within the scope of examples of this disclosure as defined by the appended claims.

1-24. (canceled)

25. A medical balloon comprising:
a balloon base having an interior surface and an exterior surface, wherein the balloon base includes a base material; and
one or more 3D printed fibers on a respective surface of the balloon base.

26. The medical balloon of claim 25, wherein the base material does not include fibers.

27. The medical balloon of claim 25, wherein the base material includes one or more fibers embedded within the base material.

28. The medical balloon of claim 27, wherein a % wt of the one or more fibers embedded within the base material is 1% to 50%.

29. The medical balloon of claim 25, wherein the one or more 3D printed fibers comprise continuous fibers.

30. The medical balloon of claim 25, wherein the one or more 3D printed fibers have been printed in a first direction on the respective surface of the balloon base.

31. The medical balloon of claim 28, further comprising:
one or more second 3D printed fibers on a surface of the one or more 3D printed fibers, wherein the one or more second 3D printed fibers have been printed in a second direction, different from the first direction, on the surface of the one or more 3D printed fibers.

32. The medical balloon of claim 25, wherein the respective surface is an exterior surface of the balloon base.

33. The medical balloon of claim 25, wherein the respective surface is an interior surface of the balloon base.

34. The medical balloon of claim 25, wherein a pattern of the one or more 3D printed fibers directionally biases collapsing of the medical balloon to a corresponding shape.

35. The medical balloon of claim 25, wherein at least one of the balloon base or the one or more 3D printed fibers include a Barium-based powder.

36. A method of manufacturing a medical balloon, the method comprising:
3D printing one or more continuous fibers on a respective surface of a balloon base of the medical balloon, wherein the balloon base includes a base material that includes one or more fibers embedded within the base material.

37. The method of claim 36, further comprising:
3D printing one or more second continuous fibers on a surface of the one or more continuous fibers in a direction different from a direction in which the one or more continuous fibers are printed.

38. The method of claim 36, wherein the respective surface is an exterior surface of the balloon base.

39. The method of claim 36, wherein the respective surface is an interior surface of the balloon base.

40. The method of claim 36, wherein 3D printing the one or more continuous fibers on the respective surface of the balloon base includes printing the one or more continuous fibers into a pattern that directionally biases collapsing of the medical balloon to a corresponding shape.

41. The method of claim 36, wherein at least one of the balloon base or the one or more continuous fibers include a Barium-based powder.

42. The method of claim 36, wherein a direction of 3D printing of the one or more continuous fibers is within 45 degrees of a load-bearing direction of the medical balloon.

43. The method of claim 36, wherein a % wt of the one or more fibers embedded within the base material is 1% to 50%.
